(19) United States
(12) Patent Application Publication
(10) Pub. No.: US 2012/0184827 A1
(43) Pub. Date: Jul. 19, 2012

(54) MINIATURE DISEASE OPTICAL SPECTROSCOPY DIAGNOSTIC SYSTEM

(76) Inventors: Shoulamit Cohen Shwartz, Atlit (IL); Meir Orenstein, Haifa (IL)

(21) Appl. No.: 13/378,916

(22) PCT Filed: Jun. 16, 2010
(86) PCT No.: PCT/IL2010/000477

§ 371 Date: Mar. 5, 2012

Related U.S. Application Data
(60) Provisional application No. 61/187,280, filed on Jun. 16, 2009.

(51) Int. Cl. G01J 3/42; A61B 5/07; A61B 10/02; A61B 5/00; A61B 1/06; A61B 5/02

(52) U.S. Cl. 600/302; 356/402; 250/339.07; 356/326; 250/341.8; 600/178; 600/479; 600/562; 600/583

(57) ABSTRACT

A miniature medical spectrometer is provided, the spectrometer comprises: a room temperature, electrically excited, solid state two photon laser generating high intensity broad wavelength; a light projection optics, projecting said generated light on biological subject; a light collection optics, collecting reflected light from said biological subject; a wavelength selector spectrally analyzing said collected light; a detector, detecting said analyzed light; and a controller analyzing the reflected spectra and calculating result indicative of the medical state of the biological subject based on said spectrum.

### FIELD OF THE INVENTION

The present invention generally relates to disease diagnostic system. In particular, the invention relates to “optical pathology” using a miniature to semiconductor based devices for infrared spectroscopic diagnostics of cancer and other maladies.

### BACKGROUND OF THE INVENTION

One of the problems that the present invention is solving is the identification of human cancer cells in real time, in-vivo or ex-vivo.

The limitations of the current practice of therapeutic excision are the inability to provide an answer, before the completion of the surgery, whether there are residual malignant cells whether the excised tumor's margins are clear or the inaccuracy of current practice (frozen section-when used) which may result in an additional surgery due to discovery of residual cancerous cells after the surgery.

In some endoscopic procedures, inability to assess-in real time, the nature of the tissue may result in missing the target tissue.

Current core needle biopsy practice is limited because the physician is not certain that the sample drawn is from the target tissue or its vicinity. Therefore multiple samples are taken.

The limitations of the current PAPS practice are: inability to have an accurate diagnostic result in real time and at point of care (gynecologist's office). In addition PAPS sensitivity and specificity, as reported in the literature, are 50% and 70-95%, respectively.

US application 20100069720A1; titled “spectroscopically enhanced imaging”; Fulghum, Stephen; et. al.; discloses systems and methods for the spectroscopic determination of the physical characteristics of the tissue under observation by an autofluorescence or other endoscope without the requirement of contacting the tissue directly. The optical probe contained in the endoscope itself is passive and may be either built into the endoscope or positioned in a biopsy channel of same. The spectroscopic information, combined with other information provided by the endoscope such as total fluorescence, improves the sensitivity and specificity of the identification of to precancerous or cancerous lesions.

US application 20090135870A1; titled “Light source based on simultaneous Two-Photon emission”; to Hayat, Alex, et. al.; discloses a semiconductor device for, e.g. target material analyzing system, produces at least 1 W/m2 two photon emission power per area, when operating at one or more temperatures greater than 20 K.

U.S. Pat. No. 5,522,870; titled “Fast changing heating-cooling device and method”; to Ben-Zion Maytal, and references therein; discloses a miniature cryogenic cooler for surgical instrument treating human skin, brain or eye—uses gas which liquefies when expanded.

The scientific literature provides a plurality of references for usefulness of optical spectroscopy for tissue diagnostics. Among these references are:

DIEM, M. X., E, S. S., & A, L. I. (1999). “Infrared Spectroscopy of Cells and Tissues: Shining Light onto a Novel Subject.”; Applied Spectroscopy. Vol 3, no 4, 1999

Haka, A. S., Shafer-peltier, K. E., Fitzmaurice, M., Crowe, J., Dasari, R. R., Feld, M. S., et al. (2005). “Diagnosing breast cancer by using Raman spectroscopy”, PNAS, vol. 102, no. 35, 12371-12376

Haka, A. S. (2006). “In vivo Margin Assessment during Partial Mastectomy Breast Surgery Using Raman Spectroscopy.”, Cancer Research, (6), 3317-3322.

Haka, A. S., Volynskaya, Z., Gardecki, J. A., Nazemi, J., Shenk, R., Wang, N., et al. (2009). “Diagnosing breast cancer using Raman spectroscopy: prospective analysis.”, Journal of Biomedical Optics, 14 (October).

Lyng, F. M., Conroy, J., Meade, A. D., Knief, P., Duffy, B., Hunter, M. B., et al. (2007). “Vibrational spectroscopy for cervical cancer pathology, from biochemical analysis to diagnostic tool.” Experimental and Molecular Pathology, 82, 121-129.

### SUMMARY OF THE INVENTION

A system according to the present invention is based on a semiconductor infrared emission source (e.g. based on 2-photons as is disclosed in U.S. patent application Ser. No. 11/987,071 Hayat et al. titled “Light Source Based on Simultaneous Two-Photon Emission” filed on Nov. 27, 2007). A hand held device or an integrated device with the semiconductor emission source and detector(s) integrated inside it is placed in proximity or in contact with the tissue and illuminates the examined site (in the examples above: excised tumor margin, margins in the excised area, target tissue in endoscopy, the target tissue of the core needle biopsy, cervix, blood vessels, internal tissues such as intestines, skin etc). The device may operate at room temperature and in the environment of surgery room. The light reflected from the tissue is collected, detected and analyzed by an adjoint processing unit, using special algorithms to determine the “finger-prints” and their classification, with the potential of accurate, possibly non-invasive, in-vivo or ex-vivo and immediate diagnosis of the presence or absence of malignant tissue or of other maladies. The device is designed for easy use in the surgery room, the Ob/Gyn practitioner, dermatologist office etc.

The present invention may be used in numerous applications, including but not limited to:

Therapeutic Excisions.

Diagnosing tumor margins during surgical oncology for residual cancerous cells. The current practice is to inspect a specimen (be it part of the removed tumor or tissue from the margins) ex-vivo or post-surgery, by a pathologist, to assess whether all cancer cells have been completely removed. Currently, “frozen section” studies are occasionally used for obtaining pathology results during surgery. However, this procedure of the art, when done during surgery, is time consuming and requires a pathologist on site. In contrast, using the current invention, results may be available during the surgery or immediately post-surgery.

Micro-Surgery and Endoscopic Procedures:

During endoscopic or minimally invasive procedures, there is a need to diagnose tissue inside the body (e.g., identify cancerous cells). The current practice in oncological micro-surgery, is to inspect a specimen ex-vivo to assess whether the specimen has cancer cells or is clear of them.

Core-needle biopsy: a needle is inserted into the body to take biopsy of tissue suspected of having some malignancy. The needle is guided to the target tissue by previously taken or “on-line” images of the area (such as ultrasonic images, X-Ray and others). On the average 6 insertions are performed in a single biopsy, as reported in the literature.

Screening and Diagnosis of Cervical Cancer.

Current practice: the gynecologist takes a sample from several locations in the cervix, called PAP smear and sends it to a lab for analysis. Results are reported after a few days.

Vulnerable Plaque.

70% of acute coronary syndromes, amongst them heart attacks are caused by “vulnerable plaque”—a hazardous type of plaque which is characterized by its lipid-rich chemical composition. These plaques are currently undetectable by the common imaging methods.

Screening and Diagnosis of Tumors and Polyps in the Digestive Tract.

Some of the diagnostics of lesions in the digestive tract, specifically in the upper part, are done using gastro-endoscope. For the large intestine, a colonoscopy is utilized. Another method is by a swallowable capsule containing a camera which travels in the digestive tract.

The system according to the present invention can be used for numerous healthcare related diagnosis applications, not limited to the applications listed below:

The system according to the present invention can be used by the surgeon during lumpectomy, nephrectomy, prostatectomy, esophagus cancer surgery, pancreatic cancer surgery, lung cancer etc. to examine the margins of the tumor and get an immediate result. This result is the indication whether all cancerous cells were removed, whether the clear margins are within the required values, or there is a need to shave additional layers in order to ensure area clear of malignant cells

The system according to the present invention can be integrated in an endoscope to be used during endoscopic and minimally invasive procedures

The system according to the present invention can be further miniaturized and integrated within the needle which is used to perform the biopsy, guiding the needle to the target tissue and possibly identify the malignant tissue

The system according to the present invention can be used by the Ob/Gyn to perform PAP smear tests in the office and obtain immediate results without the need to take a sample from the cervix and send it to the lab for analysis. (Taking a sample may be optional in case it is positive because it may be required to keep a sample when it is positive for the records and future use)

The system according to the present invention can be used by the dermatologist to assess presence or absence of suspected skin cancer.

The system according to the present invention can be integrated at the tip of an intravascular catheter; it can be used to identify vulnerable plaque.

The system according to the present invention can be embedded in a capsule, for scanning internal organs such as the esophagus, small and large intestines for suspected lesions.

The system according to the present invention can be used externally to assess glucose level in diabetic patients

Use of an infrared source which emits one or two photons, operates in room temperature, emits a wide spectrum of infrared light (e.g., near infrared, mid-infrared), can provide immediate diagnostic results. The source is made of available, inexpensive materials. Another novelty is by making the spectroscopy device small enough, enabling its insertion into the body such as in endoscopic procedures, core needle biopsy, swallow-able capsule, intravascular catheter etc.

The current invention provides a small footprint, low cost and low power consumption inspection device that allows for a variety of possible configurations, for example a hand held spectral inspection tool.

It is another aspect of the invention to provide a health monitoring device, for example glucose monitor. The monitor is preferably shaped as a wrist watch. A miniature spectrometer in the monitor directs laser light to the skin of the user and determine non-invasively the glucose level and/or level of other substances in the tissue in front of its optical window.

It is yet another aspect of the invention to provide a swallowable pill comprising a power source, a communication unit, a camera and a light source and a miniature spectrometer. In operation, the pill is swallowed and travels through the digestive track of a patient. The pill transmits to a unit outside the patient's body correlated images and at least one spectra of suspected tissue in at least one location along the digestive track.

According to one aspect of the current invention a medical spectrometer is provided, the spectrometer comprises: a solid state light source generating high intensity broad wavelength; a light projection optics, projecting said generated light on biological subject; a light collection optics, collecting reflected light from said biological subject; a wavelength selector spectrally analyzing said collected light;

a detector, detecting said analyzed light; and a housing holding said light source, said optics, said wavelength selector and said detector, wherein said housing is sized to be hand held.

In some embodiments the solid state light source is a two photon emission source.

In some embodiments the two photon emission source is a room m temperature two photon emission laser.

In some embodiments the solid state light source is capable of emitting light in the range of 2 to 5 micrometers wavelength.

In some embodiments the solid state light source is electrically excited.

In some embodiments the wavelength selector is a grating.

In some embodiments the detector is a solid state detector array.

In some embodiments the detector is a room temperature detector.

In some embodiments the spectrometer further comprises at least one waveguide for delivering light from said light source to said biological subject, wherein length of said waveguide is less than 100 cm.

In some embodiments the spectrometer further comprises at least one waveguide for delivering light from said light source to said biological subject, wherein length of said waveguide is less than 10 cm.

In some embodiments at least one of: a light projection optics; light collection optics; or wavelength selector are constructed from a sheet of IR transparent material.

In some embodiments the spectrometer is integrated into a hand held probe.

In some embodiments the spectrometer is integrated into an endoscope.

In some embodiments the spectrometer is integrated into the distal end of an endoscope.

In some embodiments the spectrometer is integrated into a vascular catheter.

In some embodiments the spectrometer is capable of determining presence of plaque on walls of blood vessels.

In some embodiments the spectrometer is integrated into a biopsy apparatus, wherein spectral data collected from said spectrometer is used for guiding the biopsy apparatus.

In some embodiments the biopsy apparatus is capable of obtaining biopsy samples, and said spectrometer is capable of real time acquisition of spectra from tissue adjacent to the biopsy-obtaining-tip of said biopsy apparatus.

In some embodiments the biopsy apparatus comprises an aspiration needle, and said spectrometer is capable of real time acquisition of spectra from aspired liquid.

In some embodiments the spectrometer is integrated into a swallowable pill.

In some embodiments the spectrometer in said pill is capable of real time acquisition of spectra from walls of the digestive track.

In some embodiments the spectrometer further comprises a controller unit, wherein said controller unit is capable of receiving data from said detector, analyzing said data and calculating result indicative of medical state of said biological subject.

In some embodiments the biological subject is human tissue, and said result is indicative of the probability of said tissue being cancerous.

In some embodiments the biological subject is human tissue and said result is indicative of the glucose level in said tissue.

In some embodiments the biological subject is human blood and said result is indicative of levels of substances such as glucose, lipids and hormones in said blood.

In some embodiments the spectral acquisition is performed in vivo.

In some embodiments the spectral acquisition is performed on extracted blood sample.

In some embodiments the spectrometer is a disposable spectrometer.

In some embodiments the light projection optics is less than 10 mm from said biological subject during acquisition of spectral data.

In some embodiments the light projection optics is in contact with said biological subject during acquisition of spectral data.

According to another aspect of the invention, a method for acquiring spectra from biological tissue is provided, the method comprises: generating high intensity, broad wavelength light in the 2 to 5 micrometer wavelength range by a room temperature, electrically excited, two photon light source; projecting said generated light onto biological subject; collecting light reflected from said biological subject; spectrally analyzing said collected light; detecting said spectrally analyzed light and generating signal indicative of optical spectrum of said detected light.

According to another aspect of the invention, a method for diagnosing tissue is provided, the method comprising: generating high intensity, broad wavelength light in the 2 to 5 micrometer wavelength range by a room temperature, electrically excited, two photon light source; projecting said generated light onto biological subject; collecting light reflected from said biological subject; spectrally analyzing said collected light; detecting said spectrally analyzed light and generating signal indicative of optical spectrum of said detected light; and calculating result indicative of medical state of said biological subject based of said signal indicative of optical spectrum of said detected light.

In some embodiments the biological subject comprises tissue cells, and calculating result indicative of medical state of said biological subject is based on differences in light reflection between cell's nucleuses and cell's cytoplasm.

In other embodiments, calculating result indicative of medical state of said biological subject is based on differences in light reflection caused by difference in fat content of said tissue.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates a miniature spectrometer-on-a-chip device in accordance with a preferred embodiment of the present invention.

FIG. 2a schematically depicts a miniature spectrometer device according to another exemplary embodiment of the invention.

FIG. 2b schematically depicts the main components of device according to an exemplary embodiment of the current invention.

FIG. 3 schematically depicts a miniature spectrometer device having its optical system primarily etched from a thin sheet of transparent material.

FIG. 4a schematically depicts a core biopsy device capable of removing a biopsy sample from tissue.

FIG. 4b schematically depicts a guided aspiration biopsy apparatus 420 according to another embodiment of the current invention.

FIG. 4c schematically depicts an optical spectroscopy probe according to an exemplary embodiment of the current invention.

FIG. 4d schematically depicts a diagnostic endoscope having a miniature spectrometer at its distal end according to an exemplary embodiment of the current invention.

FIG. 4e schematically a diagnostic endoscope according to an exemplary embodiment o the current invention.

FIG. 4f schematically depicts a vascular diagnostic catheter according to an exemplary embodiment of the current invention.

FIG. 5a shows an external view of glucose monitor according to an exemplary embodiment of the current invention.

FIG. 5b shows a cross section view of glucose monitor according to an exemplary embodiment of the current invention.

FIG. 6 schematically depicts a swallow-able spectroscopy pill according to yet another exemplary embodiment of the current invention.

### DESCRIPTION OF PREFERRED EMBODIMENTS

Before explaining at least one embodiment of the invention in detail, it is to be understood that the invention is not necessarily limited in its application to the details set forth in the following description or exemplified by the

Examples. The invention is capable of other embodiments or of being practiced or carried out in various ways.

The terms “comprises”, “comprising”, “includes”, “including”, and “having” together with their conjugates mean “including but not limited to”.

The term “consisting of has the same meaning as “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

In discussion of the various figures described herein below, like numbers refer to like parts. The drawings are generally not to scale. For clarity, non-essential elements were omitted from some of the drawing.

The system according to the claimed invention provides results in real is time easy to use, operates at point-of-care, operates at room temperature, provides full infrared spectrum, emits streams of simultaneous photon groups, provides higher signal to noise ratio, gives more accurate results, can be very small, can provide results in noisy environment, is cheap to manufacture, can be sterile and can include disposable parts, such as the emission source.

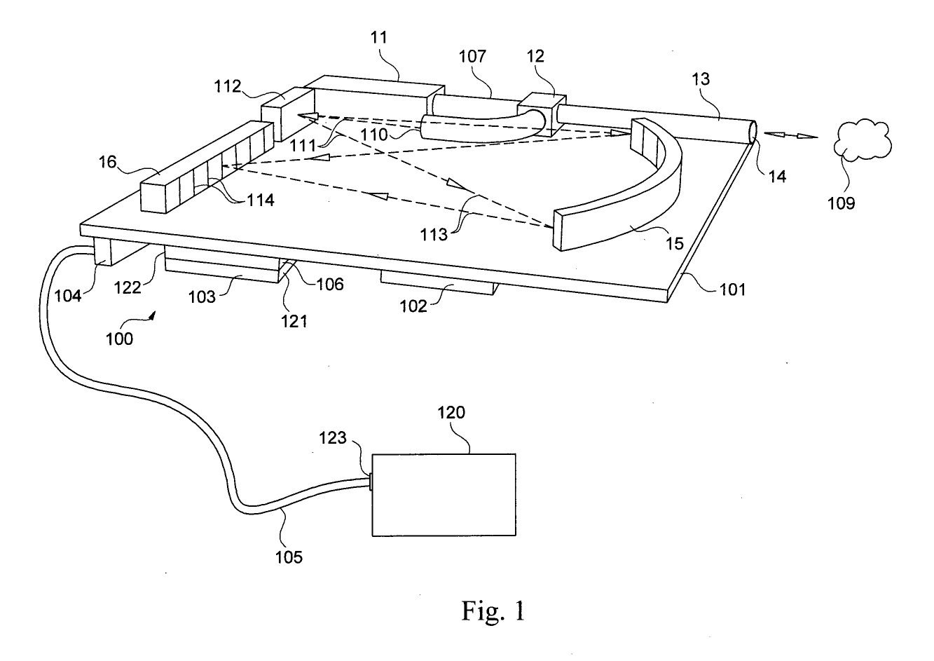

Reference is now made to FIG. 1 schematically illustrating a miniature spectrometer on a chip device 100 in accordance with a preferred embodiment of the present invention.

Miniature spectrometer device 100 comprises a carrier 101 on which electro-optical components are integrated.

Broad band light is produced by a Miniature solid-state Infrared light source 11 which emits broad spectral light. Preferably light source 11 is a two photon laser 11 capable of producing Infra Red (IR) light. Preferably, light produced by laser 11 is in the wavelength range of 2 to 5 micrometer, however different range of wavelengths may be used. For example, wavelength range of 3 to 5 micrometer may be preferred as it may probe rich spectral features of biological molecules. Optionally, wavelength range may extend to shorter wavelength such as to 1 or even 0.5 micrometer using a solid state two photon laser. In contrast to other sources of broad band light such as black body, source 11 produces high light intensity per area, and collimated light capable of being efficiently coupled into input waveguide 107. Source 11 may emit a single stream of photons, or two streams of correlated photons. For example, two streams may be chosen for improved signal to noise ratio by correlations measurements. The source may be composed of a single or a few electrically excited solid state sources.

Input light in input waveguide 107 arrives at coupler 12 and continues towards the tissue to be examined via waveguide 13 that directs the light towards the tissue, and collect the reflected light back to coupler 12.

Optical output probe tip 14 is a tip, delivering the light to and from the tested tissue 109. Tip 14 may be in contact with the tested tissue or in proximity to said tissue 109. In some applications, tip 14 is a one-use consumable element designed to provide sterility, while miniature spectrometer on a chip device 100 is capable of being reused. In other application the entire miniature spectrometer on a chip device 100 is a one-use device.

Light reflected from tested tissue 109 is collected by tip 14 and arrives at coupler 12 via waveguide 13. Coupler 12 is designed to separate the source light from the back reflected light and directs the reflected light to its output port 110. From output port 110 of coupler 12, light is spread 111 on grating 15 by a dispersing optical element such as mirror 112. Mirror 112 may be a flat or convex mirror, a lens, a lens system or combination of some of these elements. In some embodiments, spreading of light is due to the small aperture of the output port 110 of coupler 12. In some embodiments, optical element 112 is astigmatic to provide spreading of the light on grating 15, while preventing the spread of light in the direction normal to carrier 101.

Grating 15 is preferably a curved grating acting to disperse the different wavelengths of light, and focusing each range of wavelength of the wavelength dispersed light 113 on a separate element 114 of detector array 16. In some embodiments, grating 15 may be replaced with other spectral dispersing of filtering elements such as prisms, filter array or tunable spectral filter.

Detector array 16 is preferably a One Dimensional (1 D) array of detectors. Typically, each element 114 detects the reflected light in a specific range within the spectrum of interest. Detector array 16 may be composed for example of 64 elements, optionally with some separation between their detected spectrums. Alternatively, detector array 16 comprises a plurality of discrete detector elements, positioned at locations where relevant wavelengths are focused, with gaps where wavelengths irrelevant to the desired diagnosis are focused. This reduces the number of detection elements and reduces cost and power consumption. Detector array 16 preferably does not require to cryogenic temperature for its operation. Detector array may comprise indium antimonide (InSb) semiconductor detector array or a miniature bolometer array.

Preferably, integrated onto carrier 101 is electronics module 102, coupled to detector array 16 and to electric cable 105. Electronics module 102 is preferably an integrated circuit (IC) such as an Application Specific Integrated Circuit (ASIC) comprising amplifiers for the signals from elements 114 of detector 16. Electronics module 102 may provide other signal conditioning and signal processing functions such as filtering, smoothing, lock-in-amplifier function, and Amplitude to Digital Conversion (ADC) function.

Optionally, electronics module 102 comprises a plurality of ICs and/or discrete electronic elements. Electronics module 102 may also be used to control the light source 11, for example providing intensity modulation of light source 11. Modulation of light source 11 may be achieved by modulating the current powering source 11. Modulation of light source 11 may be in the form of pulses or sinusoidal. Modulation of light source 11, in combination with synchronous detection improves the Signal to Noise Ration (SNR) and allows operation of the miniature spectrometer device 100 in the presence of background light. This allows operation of the device, for example, during surgery without diming or turning off the lights.

In some embodiments, integrated to carrier 101 is a heat removal unit 121. Heat removal 121 may be a liquid or gas cooling unit, for example a miniature Joule-Thompson cooler. Preferably heat removal unit 121 comprises a Thermo Electric Cooler (TEC) 122 coupled with heat sink 103. Optional heat removal unit 121 removes heat generated by light source 11 and optionally maintain it at proper operational temperature. Additionally or alternatively heat removal unit 121 cools detector array 16 for example to maintain it at proper operational temperature. Additionally or alternatively heat removal unit 121 removes heat generated by electrical module 102. Same heat removal device may be used for cooling more than one unit of the miniature spectrometer such as electronics, light source and detector, or separate heat removal devices may be used, or some or all the units are not cooled.

Miniature spectrometer device 100 is connected to a controller unit 120, preferably using cable 105. Preferably cable 105 is connected to miniature spectrometer device 100 via connector 104, which allows replacing miniature spectrometer device 100 and reusing controller unit 120. Alternatively, cable 105 is connected to controller unit 120 at connector 123. Controller unit 120 may be powered by a battery or a rechargeable battery or may be connected to main power outlet. Controller 120 may comprise input and output devices and connectors for programming, controlling and interfacing with the controller such as display, mouse, keyboard and communication devices such as wired or wireless communication such as LAN, USB, Wi-Fi, and other public or proprietary communication protocols. Controller 120 may optionally be split to several sub units. For example, controller 120 may comprise a front end unit supplying power to, receiving data from, and controlling the spectrometer; and data processing unit for analyzing the collected data and determine the type of data. Optionally, the data processing unit may be a laptop computer, a PC, a notebook computer, a PDA, a smart phone or other computing device known in the art.

In some embodiments, for example when device 100 is part of a laboratory apparatus, miniature spectrometer device 100 and controller unit 120 are integrated into one apparatus.

In some embodiments, cable 105 allows inserting miniature spectrometer device 100 into natural or made cavity in the human body, for example using a catheter or an endoscope or a probe such as vascular catheter, urological catheter, vaginal probe or a colonoscope.

Not seen in FIG. 1 is a cover, a housing or encapsulation of device 100. For use near of within the body, said encapsulation is made of bio-compatible material and optionally can be sterilized.

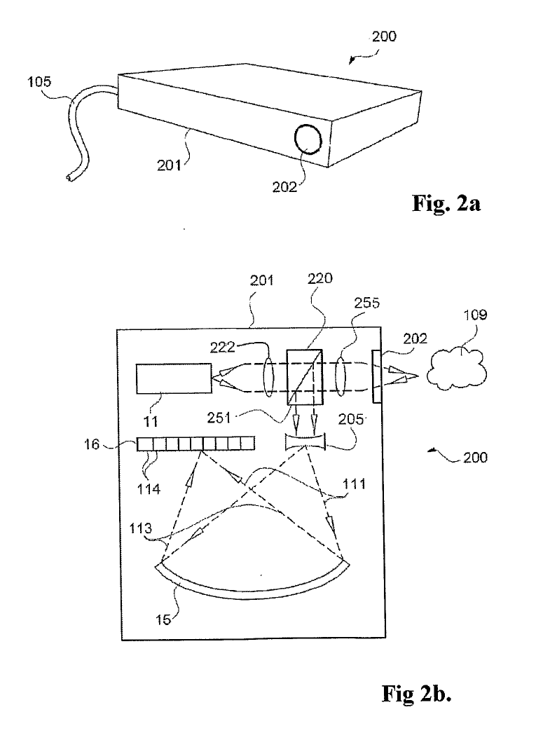

FIG. 2a schematically depicts a miniature spectrometer device 200 according to another exemplary embodiment of the invention.

Devices 100 and 200 primarily differ by their optical system used for interfacing with tissue 109. Similar components and structures are marked with like numbers and their function already disclosed.

Device 200 is seen enclosed in enclosure 201, having an optical port 202. Light exits housing 201 through optical port 202, and interacts with tissue or sample outside the housing. Light reflected from the tissue or sample enters the housing through the optical port 202, where it is analyzed and detected. Cable 105 provides electrical power to device 200, transmits data indicative of the detected light, provides control signals, and optionally carry cooling fluids.

FIG. 2b schematically depicts the main components of device 200 according to an exemplary embodiment of the current invention.

Light from source 11 is optionally collimated by optional light collimator 222 and enters coupler 220. Collimator 222 may be a lens or other optical element such as concave mirror, etc. Optionally, collimator 222 in integrated into source 11, for example as convex front face.

Coupler 220 is preferably a beam splitter directing at least some of the source light to a focusing optical element such as lens 255. In some embodiments, coupler 220 comprises a polarizer and phase retarding wave plate (not seen for drawing clarity) for efficient coupling of collimated polarized source light from source 11 to tissue 109, and efficient coupling of reflected light to output port 251 of coupler 220.

Output light 111 is spreads over grating 15 by defocusing optical element 205. Defocusing element 205 may be for example a concave lens. Alternatively, a system of lenses, or mirrors or combination thereof may be used.

Optical port 202 may be a flat optical window, transparent at the relevant wavelength. Optionally, optical port 202 is integrated with other optical components such as focusing lens 255.

In some embodiments of the invention at least some of the optical path is confined to a two dimensional (2D) waveguide in the form of thin sheet of transparent material. For example, spreading light 111, and wavelength dispersed light 113 may travel in a 2D waveguide having grating 15 etched onto it, and detector array 16 coupled to it, preferably using index matching or antireflection coating, interface or gel. In this case, defocusing elements 205 (or 112) may also be parts of the 2D waveguide. 1D waveguides 107 and 13 may also be etched in the transparent sheet. Other elements such as couplers may also be integrated or created as part of a wave-guiding sheet.

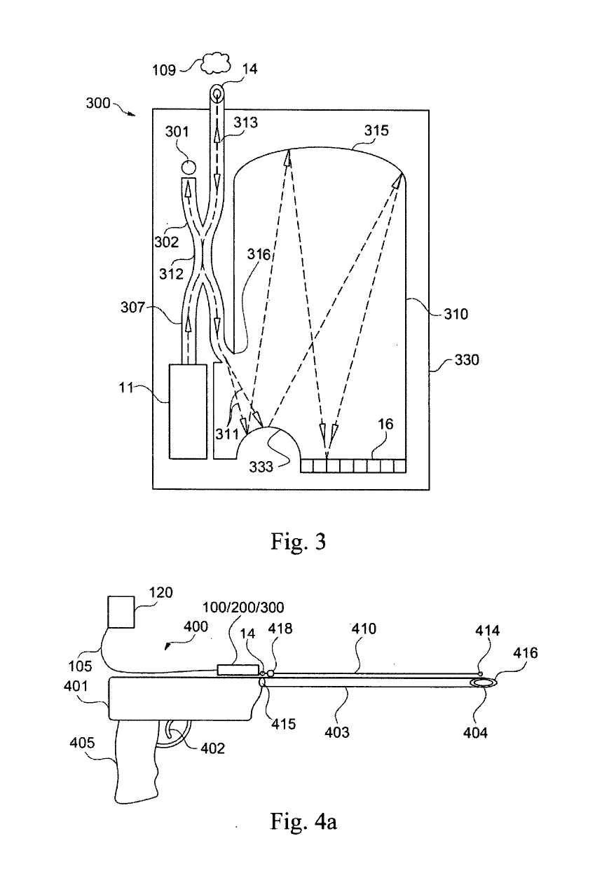

FIG. 3 schematically depicts a miniature spectrometer device 300 having its optical system primarily etched from a thin sheet of transparent material 310.

In the embodiment depicted in FIG. 3, some or all of the optical components of device 300 are integrated into one or few thin sheet of optically transparent material 300. The thickness of sheet 310 is preferably such that it confines the light in the plane of the sheet creating a 2D optical system. Confinement into 2D surface may be achieved by total internal reflection, or by reflective coating. Optionally thickness of sheet 310 is such that the confined light is limited essentially to first optical mode in the direction normal to sheet 310. Shape of sheet 310, and optical components shaped into it may be created by etching, for example lithography or laser etching or other etching methods known in the art. Preferably, sheet 310 is made of material transparent to the wavelength range used. For the preferred range of 2 to 5 micrometers, such material may be ZnSe or Si.

Sheet 310 is preferably carried on mechanical support 330. Input waveguide 307 may be etched as 1D waveguide, leading to coupler 312 and splitting to sample waveguide 313 that directs the light towards the tissue, and collect the reflected light back to coupler 312. A second port 302 of coupler 312 may be terminated by a monitoring detector 301 used for monitoring and adjusting the output power of source 11 for calibration

From output port 316 of coupler 312, light is spread 311 on grating 315 by a dispersing optical element such as mirror 333. Mirror 333 is preferably a convex etch in sheet 310. Similarly, grating 315 may also be created by etching a pattern in sheet 310.

Optionally, surface of mirror 333 and/or grating 315 are coated with highly reflecting material such as gold, Aluminum or other reflecting structure such as dielectric coating.

Sheet 310 is preferably thin enough to provide light confinement in the direction normal to its plane. For example, sheet 310 may have a thickness compatible to the light wavelength.

In some embodiments, coupler 312 is a three terminals (“Y”) coupler having no port 302. Preferably, detector array 16 is abutted to sheet 310, optionally using some index matching means such as gel, glue or coating.

Generally, coupler 12, 220 or 312 may be missing and replaced with an illumination optical path for illuminating the sample 109 with light from laser 11 and reflection optical path for collecting reflected light from sample 109 and directing it to the light spreading element such as 112 205 or 333 which spreads it on the grating.

In some embodiments, the construction of spectrometers 100, 200 or 300 is three dimensional such that optical paths may be in different layers or heights respect to carrier 101 or 330, and grating 15 may be concave or spherical.

Alternatively, to a detector array, a single detector is used in combination with a tunable filter. The filter may be situated on the optical path between laser 11 and tissue, or on the path between the tissue and said single detector.

In some embodiments light from light source is projected onto the tissue by light projection optics, and reflected light is collected by a light collection optics that is used as an input to the spectrometer, wherein light projection optics collection optics uses separate optical paths.

In some embodiments the inventive miniature spectrometer may be to small enough to be integrated into a hand held probe or to an endoscope. For example, spectrometer 100 may be as small as 50 mm by 30 mm by 15 mm. Optionally, spectrometer 100 may be as small as 20 mm by 15 mm by 5 mm or smaller.

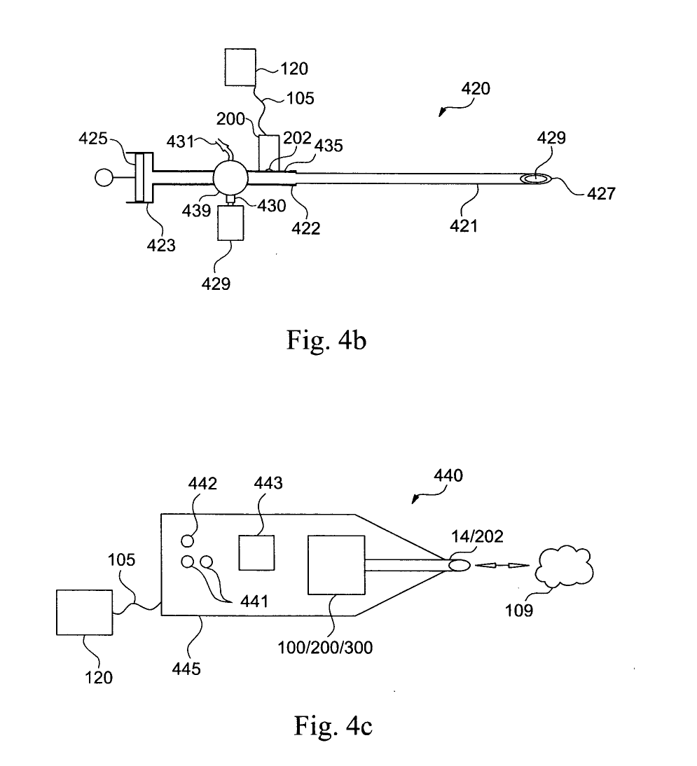

FIGS. 4a to 4e schematically depicts medical applications for miniature spectroscopy devices such as device 100, 200 or 300.

FIG. 4a schematically depicts a core biopsy device 400 capable of removing a biopsy sample from tissue. Core biopsy device 400 comprises a body 401 equipped with handle 405 and trigger 402 to trigger sample extraction from opening 404 by biopsy needle 403 having a sharp penetrating end 416 and connector 415 to connect to body 401.

Guided core biopsy device 400 further comprises a miniature spectroscopy device such as device 100, 200 or 300. The miniature spectroscopy device is attached to or integrated within body 401 of guided core biopsy apparatus 400. The miniature spectrometer is optically interfaced with the tissue to be sampled using a needle waveguide 410 ending with optical tip 414 for illuminating the tissue and collecting reflected light. Needle waveguide 410 interfaces with miniature spectrometer 100, 200 or 300 using interface 418 which interfaces the spectrometer optical port 14 (or 202) with waveguide 410.

Optionally, needle 403 and waveguide 410 with their parts are integrated into one consumable guided biopsy needle configured as one-use device. The one-use device is preferably configured for fast connection to body 401 of guided core biopsy 400. Waveguide 410 may be a hollow optical fiber or a waveguide made of IR transparent material. It should be noted that the short length of waveguide 410 reduces optical loss. Short waveguide is possible by placing spectrometer 100, 200 or 300 near the proximal end of needle 403. This is impossible to do if large size spectrometer is used, for example a Fourier Transform InfraRed spectrometer (FTIR) or large size light source such as a hot black body are used.

Spectrometer 100, 200 or 300 interfaces with controller 120 which may be integrated into body 401 or may be remotely situated, connected by cable 105.

In operation, user advances the needle 403 into the tissue while the miniature spectrometer analyzes in real time the light reflected from the tissue in location of or proximate to the location where biopsy sample may be taken.

Controller 120 report to the user about the probability of the tissue to be sampled being a target tissue. For example, controller 120 may emit an acoustic signal having volume or pitch indicative of the location of tip 414 in contact to target tissue such as cancerous tumor. Additionally or alternatively, controller 120 may comprise a display or other visual indicator for informing the user of properties of tissue near opening 404 of needle 403.

When the user discovers that opening 404 is near or at target tissue type, he may release the trigger 402 to obtain a tissue sample.

The guided biopsy apparatus according to the current invention increases the probability of obtaining the correct sample, reducing the number of needle insertions to collect specimen, reducing false negative sample, reducing cost and patient discomfort.

Needle waveguide 410 is preferably as short as needed for reducing light loss from the source to the tissue and back to the spectrometer.

Optionally, when coupler is missing from the spectrometer, needle waveguide 410 is replaced with two waveguides: one for delivering laser light, and the other for returning reflected light.

Similar configuration may apply to forceps biopsy taking apparatus where optical tip 414 is situated in proximity or within the tissue removing forceps.

FIG. 4b schematically depicts a guided aspiration biopsy apparatus 420 according to another embodiment of the current invention.

Guided aspiration biopsy 420 comprises a hollow aspiration needle 421 ending in a sharp penetrating tip 427 and connecting to the apparatus in connector 422. Needle 421 has a channel opened at orifice 429 at or near sharp 427 and leading to measurement chamber 435 in the body of guided aspiration biopsy apparatus 420.

A valve 439 connected to a suction device 423, for example a cylinder with a piston 425 allows the user to suck a fluid sample from tissue near orifice 429 into chamber 435. Sample in chamber 435 is optically probed by miniature spectroscope device 200 having optical port 202 in optical communication with fluid sample in chamber 435. Alternatively, spectrometer 200 is replaced with spectrometer 100 or 300 having its tip 14 in optical communication with fluid sample in chamber 435.

Valve 439 allows transferring the collected fluid into sample bottle 429, preferably having fast connection 430 allowing collecting a plurality of samples from plurality of locations in the tissue. Additionally and optionally, valve 439 may be set to discard the collected sample to waste 431.

In operation, the user advances the needle while drawing liquid at slow rate or intermittently. Spectrometer 200 analyzes the fluid in chamber 435 in real time. When the user determine that orifice 429 is near the target tissue, valve 439 is activated manually or automatically to collect a sample for further analysis, for example at a pathology lab.

FIG. 4c schematically depicts an optical spectroscopy probe 440 according to an exemplary embodiment of the current invention.

Probe 440 may be used for example during surgery to examine excision site after removal of a tumor for identification of cancerous tissue due to incomplete removal. Similarly, margins of removed tumor may be examined to ensure that a safety layer of healthy tissue exists around the removed tumor. Probe may be used for example for testing lymph nodes for presence of cancerous cells. The probe may also be used non-invasively for identification and classification of exposed tissue, for example testing skin lesions for cancer such as melanoma.

Probe 440 comprises a housing 445, for housing miniature spectrometer 100, 200 or 300. Tip 14 extends beyond housing 445, or window 202 on surface of housing 445 is brought to contact with or to proximity of the tissue 109 to be examined.

Control unit 120 may be integrated into housing 445 or situated remotely and connected by cable 105 to housing 445.

In some embodiments probe 440 is a self contained probe having controller 120 integrated into its body 445. In this embodiment, housing 445 may also comprise a power source such as a battery or a rechargeable battery, controlling buttons and other user inputs 441 for example such as on/off switch, activate measurement button and tissue type selector. Probe 440 may also comprise outputs such as a display 443, visual and/or audio indicator 442 and the likes.

Preferably, probe 440 is sized to be hand held. Optionally, probe 440 may be sterilized or covered with a sterile cover having a light transparent window or tip such that it may be used in an operation room. However, for external use, or to be used on removed tissue, sterilization may not be needed.

It should be noted that in the probe 440, optical path to the tissue and back is short, thus optical loss may be minimized.

Hand held probe may be made as small as few cm in size so it can be manipulated in the operation room. For example, the probe may be sized as a mobile phone.

FIG. 4d schematically depicts a diagnostic endoscope 460 having a miniature spectrometer 100, 200 or 300 at its distal end.

Endoscope 460 may be shaped and function as an endoscope of the art with an addition of optical tip 14 extending from, or window 202 exposed on its surface near its distal end.

In the depicted exemplary embodiment, distal end of shaft 460 of diagnostic endoscope 460 shown with working channel 463, illumination light source 464, camera 462 and spectroscopy tip or window 14 or 202.

It should be noted that endoscope 460 may further comprise other channels and means such as irrigation channel, optical surfaces cleaning jets, and tissue manipulation or treatment means.

Endoscope 460 may be flexible or rigid and may comprise means for navigation and positioning. Preferably, spectroscopic tip 14 or window 202 is so situates such that the part of tissue that is spectroscopically examined is within the field of view of camera 462. Optionally, tip 14 or window 202 is situated on the side wall of shaft 461. Optionally endoscope 460 comprises a plurality of tips 14 or windows 202 connected to one or to plurality of spectrometers 100, 200 or 300.

In some embodiments, controller 120 is integrated into endoscope 460. In other embodiments, parts or the entire controller 120 is remotely positioned, optionally wirelessly communicating with the endoscope.

Having miniature spectrometer 100, 200 or 300 in proximity to distal end of endoscope 460 is advantageous as it reduces loss of light in transferring light to the tissue and transferring reflected light from the tissue to the spectrometer.

FIG. 4e schematically depicts a diagnostic endoscope 480 according to an exemplary embodiment of the current invention.

In contrast to endoscope 460, miniature spectrometer 100 or 200 is located in the base 481 of endoscope 480, and connected to tip 14 with elongated waveguide 483. This embodiment is advantageous for endoscopes having thin shaft 484 such as endoscopes used in the uterus.

FIG. 4f schematically depicts a vascular diagnostic catheter 490 according to an exemplary embodiment of the current invention.

Catheter 490 comprises a flexible shaft 493 having an exposed optical port 494, preferably located on the side of the shaft close to the distal end of shaft 493. Flexible waveguide 495 communicates illumination light to port 494 and returns scattered light to spectrometer 100 or 300 in base 491 of catheter 490.

Optionally, radio-opaque mark enables locating the position, and optionally the orientation of distal end of catheter 499 using standard X-Ray fluoroscopy equipment. Other localization and orientation finding methods may be used.

In operation, shaft of catheter 490 is advanced for example in an artery to a desired location where existence or type of plaque in front of optical port 494 is determined using spectrometer 100 or 300.

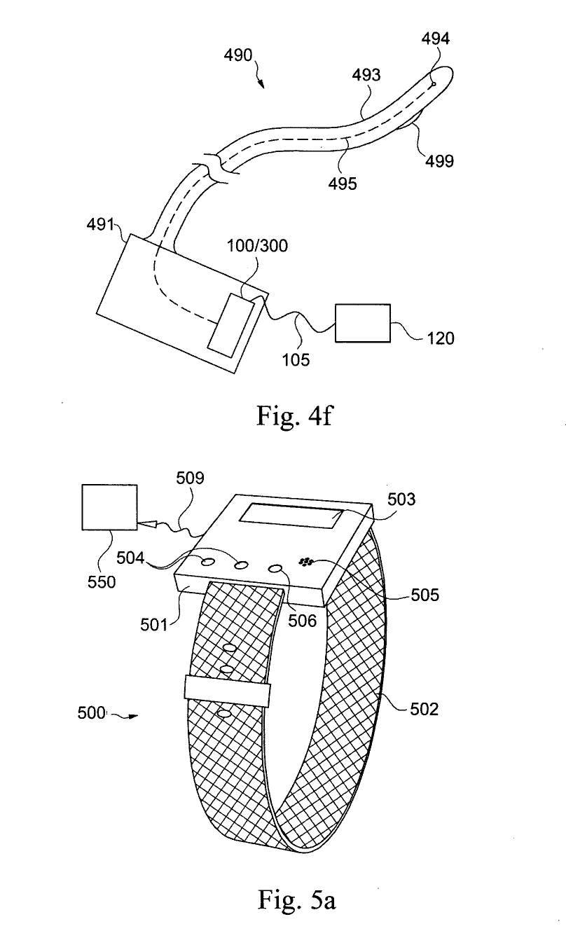

FIGS. 5a and 5b schematically depict personal spectroscopic glucose monitoring device 500 according to an exemplary embodiment of the current invention.

FIG. 5a shows an external view of monitor 500, and FIG. 5b shows a cross section of said monitor.

Monitor 500 is preferably shaped like a wrist watch having a body 501 and a wrist band 502.

Body 501 of monitor 500 comprises an optical port (not seen in this figure) on its bottom side that faces and in contact with the skin of the monitored person.

On the upper face of body 501 is a display 503 and control switches 504 for controlling the operation of monitor 500. Optional audible alarm 505 may provide acoustic alarm to alert the user of abnormal readings. Optionally, additionally or alternatively, vibration may be used as alarm.

Optionally, monitor 500 may act as a normal watch showing time, date, etc.

Optionally, monitor 500 is attached to the body at location other than the wrist, for example on the abdomen or the arm or leg. In some embodiments strap is missing and adhesive is used.

Preferably, monitor 500 comprises a wireless communication unit 508 for wirelessly communicating 509 with external unit 550. In these cases monitor 500 may not comprise display or controls except for example an optional on/off switch and/or optional indicator 506 such as on/off indicator and/or low battery indicator. In some embodiments, external unit 550 may comprise an insulin pump, for example external or implanted insulin pump. Using an insulin pump with an optical glucose monitor according to the current invention enables for example a continues and optionally fully automatic control of glucose levels in diabetic patients.

External unit 550 may be an insulin pump, internally implanted or external to the user.

External unit 550 may provide some of the functions of controller 120, for example analyzing spectra obtained by spectrometer 100, 200 or 300 to determine and log glucose level. External unit 550 may be in constant communication with monitor 500 or communicate periodically or on demand. External unit 550 may be a smart phone, a computer or a specifically made device made for communicating with monitor 500.

Monitor 500 is powered by a replaceable or rechargeable battery 510.

Monitor 500 may optionally, additionally or alternatively monitor other bodily functions and levels of chemicals other than glucose, for example oxygenation level, hormonal levels.

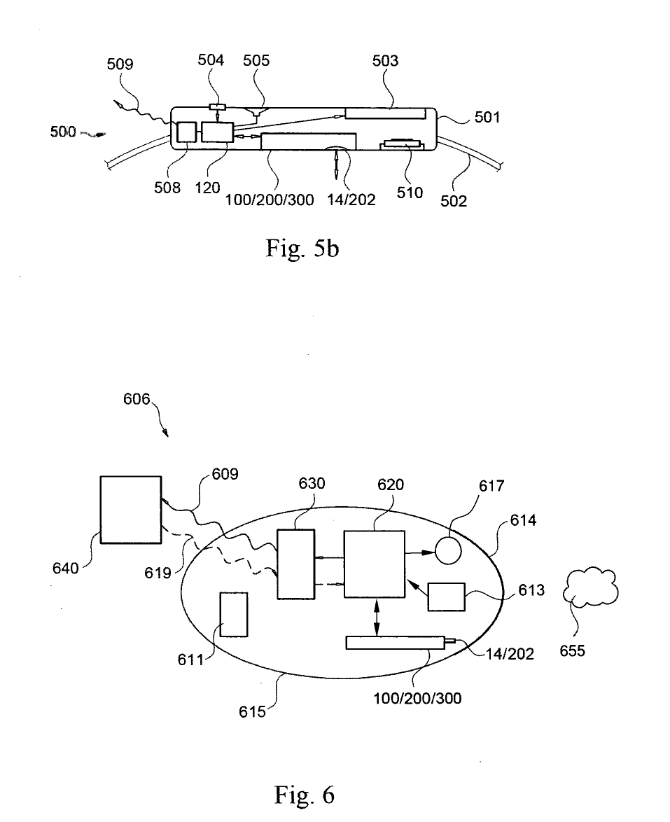

FIG. 6 schematically depicts a swallow-able spectroscopy pill 606 according to yet another exemplary embodiment of the current invention.

Pill 606 comprises a body 615 sized to be swallowed by a human patient and pass through his digestive track.

Body 615 comprises a transparent dome 614. Camera 613 and illumination light source 617 are positioned behind dome 614 for providing images of digestive track tissue 655 in front of said dome. Light source 617 illuminates the tissue 655 and camera 613 images the light reflected from the tissue 655.

Controller 620 receives image data from camera 613 and transmits said image data using wireless communication unit 630 to control unit 640 over RF link 609.

Control unit 640 preferably comprises a computer such as a PC, laptop, or a notebook computer or other computer having a display and user interface such as a keyboard or a mouse.

Once a user identifies a suspected tissue 655 from said camera's image, he uses user input on control unit 640 to communicate via optional link 619 and wireless communication unit 630 a command, commanding controller 620 to activate miniature spectrometer 100, 200 or 300 within body 615 to acquire a spectra of said suspected tissue 655.

Alternatively, spectroscopic data is acquired and transmitted automatically or periodically. Optical port 14 or 202 of miniature spectrometer 100, 200 or 300 is preferably oriented such that IR source light is directed towards tissue 655 and reflected IR light is reflected at least in part to said optical port 14 or 202.

Additionally or alternatively, acquiring spectra may be triggered automatically, for example by an image processing unit, identifying that suspected tissue is in front of the spectrometer.

Acquired spectra from tissue 655 may be analyzed by unit 640 to determine the probability of said tissue being cancerous or inflamed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A medical spectrometer comprising:
a solid state, electrically excited, two photon emission light source generating high intensity broad wavelength infrared light;
a light projection optics, projecting said generated light on biological subject;
a light collection optics, collecting reflected light from said biological subject;
a wavelength selector spectrally analyzing said collected light;
a detector, detecting said analyzed light; and
a housing holding said light source, said optics, said wavelength selector and said detector, wherein said housing is sized to be hand held.

2. (canceled)

3. The spectrometer of claim 1, wherein said two photon emission source is a room temperature two photon emission source.

4. The spectrometer of claim 1 wherein said solid state light source is capable of emitting light in the range of 2 to 5 micrometers wavelength.

5. (canceled)

6. The spectrometer of claim 1 wherein said wavelength selector is a grating.

7. The spectrometer of claim 1 wherein said detector is a room temperature solid state detector array.

8. (canceled)

9. The spectrometer of claim 1 and further comprising at least one waveguide for delivering light from said light source to said biological subject, wherein length of said waveguide is less than 100 cm.

10. (canceled)

11. (canceled)

12. The spectrometer of claim 6, wherein at least one of: a light projection optics; light collection optics; or wavelength selector are constructed from a sheet of IR transparent material.

13. The spectrometer of claim 1 wherein said spectrometer is integrated into a hand held probe.

14. The spectrometer of claim 1 wherein said spectrometer is integrated into an endoscope.

15. The spectrometer of claim 10, wherein said spectrometer is integrated into the distal end of an endoscope.

16. The spectrometer of claim 1 wherein said spectrometer is integrated into a vascular catheter, and wherein said spectrometer is capable of determining presence of plaque on walls of blood vessels.

17. (canceled)

18. The spectrometer of claim 1 wherein said spectrometer is integrated into a biopsy apparatus capable of obtaining biopsy samples, wherein spectral data collected from said spectrometer is used for guiding the biopsy apparatus and said spectrometer is capable of real time acquisition of spectra from tissue adjacent to the biopsy-obtaining-tip of said biopsy apparatus.

19. (canceled)

20. The spectrometer of claim 1 wherein said spectrometer is integrated into an aspiration needle biopsy apparatus capable of obtaining liquid samples, wherein spectral data collected from said spectrometer is used for guiding the biopsy apparatus, and said spectrometer is capable of real time acquisition of spectra from aspired liquid.

21. The spectrometer of claim 1 wherein said spectrometer is integrated into a swallowable pill and wherein said spectrometer is capable of real time acquisition of spectra from walls of the digestive track.

22. (canceled)

23. The spectrometer of claim 1 and further comprising a controller unit, wherein said controller unit is capable of receiving data from said detector, analyzing said data and calculating result indicative of medical state of said biological subject.

24. The spectrometer of claim 16 wherein said biological subject is human tissue, and said result is indicative of the probability of said tissue being cancerous.

25. (canceled)

26. The spectrometer of claim 16 wherein said biological subject is human blood and said result is indicative of levels of substances such as glucose, lipids and hormones in said blood.

27. The spectrometer of claim 1 wherein said spectral acquisition is performed in vivo.

28. The spectrometer of claim 26 claim 1 wherein said spectral acquisition is performed on extracted blood sample.

29. The spectrometer of claim 1 wherein said spectrometer is a disposable spectrometer.

30. (canceled)

31. The spectrometer of claim 1 wherein said light projection optics is in contact with said biological subject during acquisition of spectral data.

32. A method for acquiring spectra from biological tissue comprising:
generating high intensity, broad wavelength light in the 2 to 5 micrometer wavelength range by a room temperature, electrically excited, two photon light source;
projecting said generated light onto biological subject;
collecting light reflected from said biological subject;
spectrally analyzing said collected light;
detecting said spectrally analyzed light and generating signal indicative of optical spectrum of said detected light.

33. A method for diagnosing tissue comprising:
generating high intensity, broad wavelength light in the 2 to 5 micrometer wavelength range by a room temperature, electrically excited, two photon light source;
projecting said generated light onto biological subject;
collecting light reflected from said biological subject;
spectrally analyzing said collected light;
detecting said spectrally analyzed light and generating signal indicative of optical spectrum of said detected light; and
calculating result indicative of medical state of said biological subject based of said signal indicative of optical spectrum of said detected light.

34. The method of claim 27 wherein said biological subject comprises tissue cells, and calculating result indicative of medical state of said biological subject is based on differences in light reflection between cell's nucleuses and cell's cytoplasm.
